(19) United States
(12) Patent Application Publication
(10) Pub. No.: US 2004/0102652 A1
(43) Pub. Date: May 27, 2004

(54) Optically active2-aminotetralin derivatives, the processes for the preparation thereof and the therapeutical use of pharmaceutical compositions containing them

(76) Inventors: Gabriele Amari, Parma (IT); Maurizio Del Canale, Parma (IT); Roberta Razzetti, Parma (IT); Pier Alessandro Monici Preti, Parma (IT); Ivano Rondelli, Parma (IT)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,894

(22) PCT Filed: May 8, 2001
(86) PCT No.: PCT/EP01/05212

(30) Foreign Application Priority Data
May 12, 2000 (IT) MI2000A001053

(51) Int. Cl. A61K 31/135; A61K 31/24; C07C 215/86

(52) U.S. Cl. 564/428; 560/136; 514/540; 514/657

(57) ABSTRACT

The use of the optically active forms of 5,6-dihydroxy-2-methylaminotetralin and acyl esters thereof as medicaments for cardiovascular diseases a process for the preparation thereof and their use in pharmaceutical compositions.

### SUMMARY OF THE INVENTION

The present invention relates to the enantiomers of the compounds of formula (I)

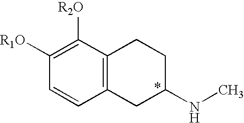

wherein R₁ and R₂ are hydrogen or C₁-C₄ acyl groups, in particular isobutyroyl, and the pharmaceutically acceptable salts thereof, as therapeutical agents.

In particular the invention relates to the use of (−)-(S) and (+)-(R)-5,6-diisobutyroyloxy-2-methylaminotetralin for the preparation of pharmaceutical compositions for the therapy of some cardiovascular diseases.

The enantiomers of the invention preferably have an optical purity ranging from 95 to 100%.

### BACKGROUND OF THE INVENTION

(±)-(R,S)-5,6-Diisobutyroyloxy-2-methylaminotetralin, hereinafter referred to as CHF 1035, has been first described in GB 2,123,410 among a series of aminotetralin derivatives disclosed as potential antibronchospastic agents. CHF 1035, after administration through different parenteral routes (oral, transdermal, and the like) is quickly and completely converted by plasma and tissular esterases into its desesterified form, namely (±)-5,6-dihydroxy-2-methylaminotetralin, indicated hereinafter with the experimental code CHF 1024.

Following extensive studies on the receptor activity profile of CHF 1024, which is the pharmacologically active moiety, it has been claimed in WO 96/29065 the use of CHF 1035 and of its metabolite for the treatment of the cardiac disorders, in particular for the therapy of congestive heart failure.

The applicant therein reported on the clinical results after single administration of racemic CHF 1035 in the form of tablets at three dose levels, i.e. 5, 10 and 15 mg, in patients with a moderate congestive heart failure (class NYHA II-III).

CHF 1024 proved indeed to be capable of selectively stimulating α₂-adrenergic and DA₂-dopaminergic pre-synaptic receptors; stimulating activities on β₂, DA₁ and β₁ receptors were observed only at concentrations 5 to 400 times higher, whereas the agonist activity on α₁ receptor was negligible.

Said receptorial profile mainly results in a vasodilating action, without reflected increase in the release of catecholamines (adrenalin and noradrenalin) and in the heart rate.

The most recent trends in the therapy of cardiac failure attribute great importance to the reduction of peripheral resistances and heart rate and to the modulation of the neurohumoral system. In particular, the use of drugs inducing remarkable and, above all, long-lasting reduction of heart rate as well as decrease in plasma catecholamines should be preferred.

### DISCLOSURE OF THE INVENTION

It has now been found that the CHF 1024 optical antipodes have significantly different pharmacodynamic profiles from each other, which makes it possible to envisage different therapeutical uses for said compounds as well as for the corresponding pro-drugs which are able to release them in vivo quickly and quantitatively, such as the catechol group acyl derivatives. The (−)-(S) enantiomer of CHF 1024—-hereinafter referred to with the experimental—code CHF 1870—has indeed been found to have affinity and selectivity toward DA₂ and α₂ receptors, both in binding studies and in isolated tissue preparations such as rabbit rectococcigeus muscle and rabbit ear artery, which are particularly rich in such receptors, said activity being significantly higher than that of the (+)-(R) enantiomer—hereinafter referred to as CHF 1869—and also remarkably higher compared with the racemate. In an in vivo model, after administration through intravenous infusion in anaesthetized normotensive rats, CHF 1870 induced a hypotensive response slightly lower but remarkably longer-lasting than that of the racemate as well as CHF 1869 which, conversely, induced a more rapid but less lasting response. Furthermore, the subcutaneous administration of CHF 1870 to spontaneously hypertensive conscious rats for 7 days induced a more marked reduction of heart rate than an equivalent dose of the racemate, whereas, in the same experimental model, CHF 1869 induced no reduction of heart rate, although causing comparable hypotensive effects.

Said observations coherently reflect both the greater selectivity of the (−)-(S) form for pre-synaptic receptors and the relatively higher contribution of the β₂ receptors stimulation in the activity of the (+)-(R) form, as it can be evinced by the results of the binding studies reported in Table 1 (Example 5).

In virtue of said pharmacodynamic and kinetic profiles, CHF 1870 and the corresponding acyl derivatives, particularly the diisobutyroyl ester, hereinafter referred to with the experimental code CHF 1810, are suitable for the preparation of pharmaceutical compositions to be used in the treatment of hypertension and heart failure, particularly congestive heart failure. As mentioned above, the more recent trends, especially in the therapy of the latter disease, give great value to the use of medicaments having a hemodynamic-neurohumoral profile characterized by reducing heart rate while inducing long-lasting inhibition of the sympathic-adrenergic activity (Ferrari R. Eur. Heart J. 1999, 20, 1613-1614). As a consequence, a better modulation of said parameters can be obtained by administration of drugs characterized by more selective receptor activity and longer-lasting action, such as is CHF 1810. Said kinetic feature would provide, on the one hand, a simpler dosage regimen for the drug, with a single daily administration, which in its turn involves remarkably better compliance, in particular in the case of patients under polytherapy.

In a preferred embodiment of the invention, CHF 1810 is employed in the preparation of pharmaceutical compositions for the treatment of patients affected by congestive heart failure and a concomitant sympathetic nervous system hyperactivity especially belonging to the higher NYHA functional classes (Criteria Committee of the New York Heart Association. Nomenclature abd Criteria for Diagnosis of Diseases of the Heart and Great Vessels; 7^{th} Ed., 1973). In clinical practice indeed, the severity of symptoms and functional capacities are gauged by a subjective scale, first introduced by the New York Heart Association (NYHA), in which the patients are assigned to 1 of 4 functional classes: patients may have symptoms of heart failure at rest (class IV); on less than ordinary exertion (class III); on ordinary exertion (class II); or only at levels that would produce symptoms in normal individuals (class I).

On the other hand experimental data also support the early treatment (NYHA class I) of transdermal CHF 1810 in the course of heart failure to slow or avoid the cardiac remodeling process.

CHF 1869 and the corresponding acyl derivatives, particularly the isobutyroyl ester, hereinafter referred to as CHF 1800, due to the relatively higher contribution of β₂ receptors in their action and to a more prompt response resulting in a more rapid onset of the therapeutical effect, may be used both in the treatment of acute hypertensive crisis and in some pathologies characterized by poor vascularization of the lower limbs, such as peripheral obliterans arteriopathy. In principle, the use of said compounds may be envisaged whenever a prompt decrease in the peripheral vascular tone is necessary. It has indeed been found that upon oral administration of the racemate (CHF 1035) the effects related to the pharmacodynamic activity of the dextro form are masqued. This has to be ascribed to the fact that, after oral administration of the racemate, the area under the curve which represents the plasma levels in time (AUC) of the dextro form, due to the particular kinetic behaviour, is about one half that of the laevo form. Therefore, CHF 1800 may be valuable for use in the therapy of the pathologies cited above.

The administration of the compounds of the invention may be carried out through any route, preferably through the oral route.

For the oral administration, the compounds can be formulated in solid or liquid preparations, preferably in tablets, by using the additives and excipients conventionally used in the pharmaceutical technique.

More preferred is the use of CHF 1810 in the form of patches for transdermal use, adapted for administering the active ingredient once a day at a daily dosage comprised from 0.01 mg/kg/day to 1 mg/kg/day, preferably from 0.02 mg/kg/day to 0.5 mg/kg/day, more preferably from 0.03 mg/kg/day to 0.15 mg/kg/day. These activities are equivalent to unit daily dosages from 2.5 mg to 50 mg, preferably from 5 mg to 20 mg. Said formulations are indeed the only ones capable of mimicking the administration through infusion; the desired levels of circulating drug are in fact attained gradually, which makes it possible to reduce the risk of abrupt pressure drop. For the transdermal use, CHF 1810 turns out to be more suitable than racemic CHF 1035 from the manufacturing stand point as well.

The current transdermal systems (patches) are indeed generally constituted of: i) an outer backing layer which is a protective barrier preventing loss of drug from the outer surface of the patch; ii) the drug reservoir which is made of a polymeric matrix, either hydro- or lipophilic in which the drug is moulded; iii) optionally, a special membrane which controls the release of the drug from the reservoir; iv) an adhesive layer which effectively attaches the patch to the skin; v) a protective liner over the adhesive layer which is removed before applying the patch.

The process of moulding usually occurs by pre-dissolving the drug into the matrix at 40-60° C., followed by casting and drying.

Racemic CHF 1035 shows a complicate profile of crystal modifications. The diffraction studies contributed to identify three different polymorphs, (forms I, II and III), and to put into evidence that form I shows two remarkable structural rearrangements, reversibly taking place between room temperature and 65° C., and within the range 65-86° C., respectively.

It is possible that, after dissolution in the adhesive matrix, interconversion occurs between the two distinct crystal structures belonging to the form I subsystem of racemic CHF 1035. As a final result of the process, a different crystalline form of the drug could form, endowed with different physical properties and thus stability and bioavailability performances.

On the contrary, CHF 1810 does not show any structural rearrangement below 100° C. Therefore, it can be incorporated in the adhesive matrix without any risk of polymorphic transition.

The enantiomers of 5,6-dihydroxy-2-methylaminotetralin as well as those of the corresponding acyl derivatives can be prepared with conventional techniques starting from the racemic compounds by fractional crystallization of the addition salts thereof with suitable optically active acids. The racemic compounds can in their turn be prepared as disclosed in GB 2,123,410 or according to the teaching reported in WO 95/29147.

Alternatively, the (−)-(S)- and (+)-(R)-enantiomers of CHF 1024 may be prepared by using enantioselective syntheses.

In particular, they can be prepared according to the process 3 of WO 95/29147 by stereoselectively reducing 5-(2,3-dialkoxyphenyl)-3-alkoxycarbonylamino-2,5-dihydrofuran-2-one which is one of the key intermediates of the process. However, step 5 of said process, which involves the direct reduction of the alkylcarbamic group, in particular methoxycarbonylamino, to alkylamino group, in particular methylamino, greatly reduces the overall yield. Many attempts to improve the yield of the reduction reaction of 2-methoxycarbonylamino-5,6-dimethoxy-tetralin by means of LiAlH₄ in THF, which is 52%, were unsuccessful.

It has now been found, and it is a further object of the present invention, that when the alkylcarbamic derivative is first subjected to N-methylation and the resulting N-methylalkylcarbamate is subsequently hydrolysed and deprotected to give 5,6-dihydroxy-2-methylaminotetralin, the overall process yield may be significantly improved. The yield of the N-methylation step is in fact higher than 85%, while that of the hydrolysis/deprotection is approximately 80-90%, either when carried out simultaneously or sequentially.

The necessary steps are reported in detail in the Scheme below and are the following:

a) condensation of 4-(2,3-dialkoxyphenyl)-2-ketobutenoic acid with a short chain (C₁-C₄) alkyl carbamate to give 5-(2,3-dialkoxyphenyl)-3-alkoxycarbonylamino-2,5-dihydrofuran-2-one;

b) catalytic reduction of the condensation product in stereoselectivity conditions to give one of the two enantiomers of 4-(2,3-dialkoxyphenyl)-2-alkoxycarbonylaminobutyric acid;

c) intramolecular cyclization to give one of the two enantiomers of 5,6-dialkoxy-2-alkoxycarbonylamino-1-tetralone;

d) reduction of the keto group to give one of the two enantiomers of 5,6-dialkoxy-2-alkoxycarbonyl-aminotetraline, preferably by catalytic hydrogenation in the presence of strong acids;

e) N-methylation of 5,6-dialkoxy-2-alkoxycarbonylaminotetralin to give one of the two enantiomers of N-methyl-5,6-dialkoxy-2-alkoxycarbonylaminotetralin, for example with methyl iodide and sodium hydride in tetrahydrofuran;

f) hydrolysis of the alkoxycarbamic group and deprotection of the catechol group to give 5,6-dihydroxy-2-methylamino-tetralin. Said reaction may be carried out either in a single step using, for example, 48% hydrobromic acid, or in two subsequent steps according to known techniques.

The corresponding acyl derivatives can be prepared by acylation of the catechol hydroxyls with known techniques.

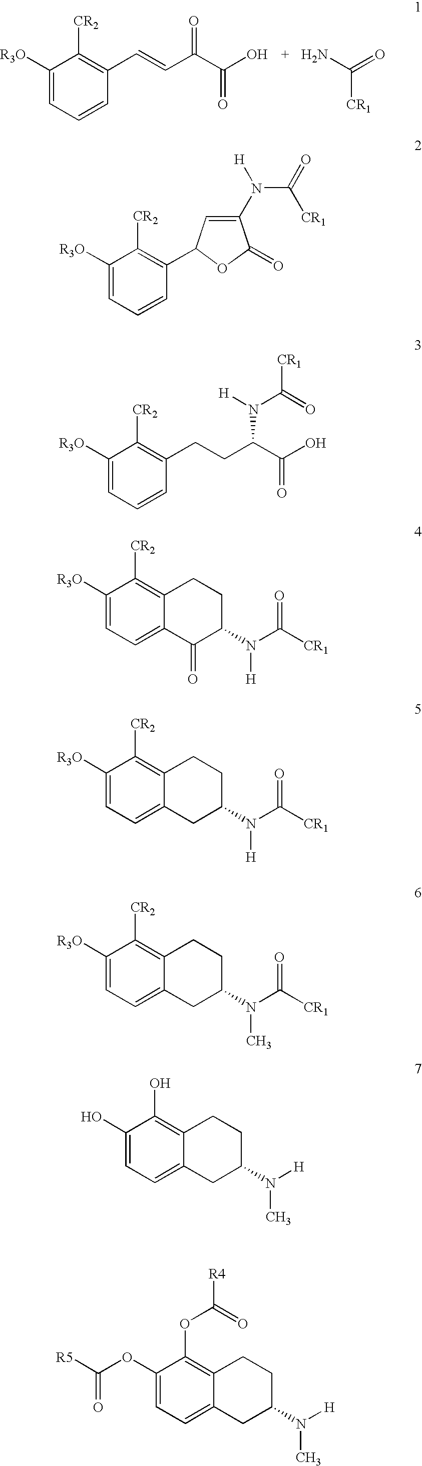

R₁=methyl; R₂ and R₃=C₁-C₄ alkyl; R₄ and R₅=C₁-C₃ alkyl

The invention is illustrated in detail in the following examples.

### EXAMPLE 1

### Preparation of (+)-(R)-5,6-disobutyroyloxy-2-methylaminotetralin hydrochloride (CHF 1800) by Resolution Through Fractional Crystallisation

### a) Preparation of (+)-(R)-5,6-diisobutyroyloxy-2-methylaminotetralin(−)-L-dibenzoyl-tartrate

80 g of (±)-5,6-diisobutyroyloxy-2-methylaminotetralin hydrochloride (CHF 1035), prepared according WO 95/29147, are dissolved in 650 ml of an aqueous solution containing a stoichiometric amount of sodium bicarbonate. The solution is extracted with chloroform (3×700 ml). The opalescent organic phases are combined, washed with sodium chloride saturated water (2×700 ml), dried over sodium sulfate and evaporated under vacuum at 35° C.

The resulting orange oil is taken up with 600 ml of an ethanol:water 1:1 v/v solution containing a stoichiometric amount of (−)-L-dibenzoyltartaric acid monohydrate (81.6 g). The mixture is heated to ebullition until complete dissolution, then the product is left to precipitate for 24 hours at room temperature. Mother liquors are kept separately. The resulting solid is recrystallized from boiling ethanol:water 2:1 v/v to obtain a white crystalline product with melting point 205-206.5° C., which is dried under vacuum at 45° C.

### b) Preparation of (+)-(R)-5,6-diisobutyroyloxy-2-methyl-aminotetraline hydrochloride

33 g of (+)-(R)-5,6-diisobutyroyloxy-2-methylaminotetralin (−)-L-dibenzoyl-tartrate are suspended in 200 ml of chloroform, then 170 ml of 5M ether hydrochloric acid are added. The resulting clear solution is stirred at room temperature for 1 hour, then 300 ml of ethyl ether are added to obtain a white crystalline precipitate. The precipitate is filtered and the solid residue is treated for 15 min in 250 ml of hot acetone, then cooled, filtered and dried under vacuum at 60° C.

m.p.=205-208° C.; [α]_{D}=+48.5 (c=0.98%, H₂O); e.e. (GC-MS): 99.6%

### EXAMPLE 2

### Preparation of (−)-(S)-5,6-diisobutyroyloxy-2-methylaminotetralin hydrochloride (CHF 1810) by Resolution Through Fractional Crystallization

### a) Preparation of (−)-(S)-5,6-diisobutyroyloxy-2-methylaminotetralin(+)-D-dibenzoyl-tartrate

The mother liquors from the step b) of Example 1 are evaporated to dryness under vacuum at 40° C. The residue is taken up with 1300 ml of methylene chloride and repeatedly washed with 600 ml of a 0.3M sodium bicarbonate aqueous solution to obtain a basic solution. The organic phase is dried over sodium sulfate and evaporated under vacuum at 35° C.

The resulting orange oil is added to 1000 ml of an ethanol:water 2:1 v/v solution containing 27 g of (+)-D-dibenzoyltartaric acid. The mixture is refluxed to complete dissolution, then left to crystallize at room temperature for 24 hours. The residue is recrystallized from boiling ethanol:water 2:1 to obtain a crystalline product with melting point 204-206° C., which is dried under vacuum at 45° C.

### b) Preparation of (−)-(S)-5,6-diisobutyroyloxy-2-methylaminotetraline hydrochloride

The procedure described in Example 1b) is followed.

M.p.=206-208° C.; [α]_{D}=−48.2 (c=0.98%, H₂O); e.e. (GC-MS): 99.2%.

### EXAMPLE 3

### Preparation of (−)-(S)-5,6-diisobutyroyloxy-2-methylaminotetralin (CHF 1810) Through Enantioselective Synthesis

### a) Preparation of 3-methoxycarbonylamino-5-(2,3-dimethoxy-phenyl)-2,5-dihydrofuran-2-one

270 g (1.14 mol) of 2-keto-4-(2,3-dimethoxyphenyl)-3-butenoic acid are dissolved in 2650 ml of toluene, then 13.4 g (0.07 mol) of p-toluenesulfonic acid and 133.8 g (1.78 mol) of methyl carbamate are added under stirring. The mixture is refluxed for 4 hours, removing the formed water by azeotropical distillation. The mixture is cooled, the turbid solution is filtered and the filtrate is evaporated under vacuum. The residue is taken up with ethyl ether (1200 ml), the solid is filtered, washed with petroleum ether and dried under vacuum at 60° C.

Yield: 313 g (94%); TLC (CH₂Cl₂, 100%): Rf=0.34.

### b) Preparation of (+)-2-methoxycarbonylamino-4-(2,3-dimethoxy-phenyl-butyric acid

5 g (17 mmols) of a suspension of 3-methoxycarbonylamino-5-(2,3-dimethoxy-phenyl)-2,5-dihydrofuran-2-one and 15 micromols of rhodium (R,R)-EtDiPhos(COD)OTs complex in 70 ml of previously degassed methanol are hydrogenated in a Parr apparatus (P=30 psi, T=20°) under stirring for two hours. The mixture is filtered and the solution is evaporated under vacuum.

Yield: 5 g; Conversion: 95%; e.e.(NMR): 92%.

### c) Preparation of (−)-5,6-dimethoxy-2-methoxycarbonylamino-1-tetralone

9.2 g (0.03 mol) of (+)-2-methoxycarbonylamino-4-(2,3dimethoxyphenyl)-butyric acid in 185 ml of methylene chloride, under nitrogen atmosphere, are cooled to 0° C., then 7.3 g (0.035 mol) of PCl₅ are added. The mixture is stirred at 0-5° C. for an hour, then added with 9.6 g (0.037 mol) of SnCl₄ and stirred at 0° C. for a further 30 minutes, then for 4 hours at room temperature. The mixture is then poured into ice-water, stirring for 20 minutes, then extracted with methylene chloride (3×300 ml). The combined organic phases are washed with water (4×300 ml), dried over sodium sulfate and evaporated to dryness under vacuum.

The solid residue is taken up with ethyl ether (30 ml) and petroleum ether (300 ml); the mixture is left to stand overnight, then filtered and dried under vacuum at 30° C.

Yield: 5.2 g (60%); e.e.(NMR)=90%.

### d) Preparation of (−)-2-methoxycarbonvlamino-5,6-dimethoxy-tetralin

3.3 g (29 mmol) of triethylsilane are added to a solution of (−)-5,6dimethoxy-2-methoxycarbonylamino-1-tetralone (2.0 g, 7 mmol) in 18 ml of BF₃.(Et₂O), under nitrogen atmosphere. The mixture is stirred at room temperature for 24 hours, then a NaHCO₃ saturated solution is slowly added to pH 8. The mixture is extracted with ethyl ether (3×200 ml), the organic phases are combined, dried over sodium sulfate and evaporated under vacuum. The solid residue is dissolved in 200 ml of methylene chloride, 4 g of silica gel are added. The mixture is stirred for 30 min, then filtered and the filtrate is evaporated to dryness under vacuum at 30° C.

Yield: 1.24 g (65%); e.e.(NMR)=95%.

### e) Preparation of (−)-N-methyl-5,6-dimethoxy-2-methoxycarbonyl-amino-tetralin

A solution of (−)-2-methoxycarbonylamino-5,6-dimethoxytetralin (10 g, 37.7 mmol) in 100 ml of dry THF is added drop by drop during 15 minutes to a suspension of NaH (1.7 g, 56.6 mmol—80% in mineral oil) in 200 ml of dry THF. The resulting suspension is stirred for 1 hour, then 10 g of CH₃I (69 mmol) dissolved in 50 ml of dry THF are added drop by drop in 10 minutes and stirring is continued for a further 8 hours at room temperature. The solution is evaporated under vacuum. The resulting oil is dissolved in 300 ml of CHCl₃, washed with 100 ml of 1N HCl and then with 100 ml of water; subsequently it is dried over dry sodium sulfate and evaporated under vacuum. The residue is purified by silica gel chromatography (32-63 micron) using as eluent petroleum ether:ethyl acetate 7:3 v/v to obtain a colorless oil which solidifies after some time.

Yield: 9.4 g (88%); TLC (petroleum ether:ethyl acetate 7:3 v/v): Rf=0.4.

### f) Preparation of (−)-5,6-dimethoxy-2-methylaminotetralin hydrochloride

A solution of (−)-N-methyl-5,6-dimethoxy-2-methoxycarbonylaminotetralin (9.4 g, 33.3 mmol) in 400 ml of CH₃OH is added with 50 g of 80% KOH in 40 ml of water and the mixture is refluxed for 48 hours. Most solvent is evaporated off under vacuum, 200 ml of water are added and the mixture is extracted with chloroform (3×150 ml); the organic phases are combined, dried over dry sodium sulfate and evaporated to dryness. The residual oil is taken up with 200 ml of acetone and 3.3 ml of 37% HCl are added under stirring. Crystallization of the corresponding hydrochloride almost immediately starts; stirring is continued for a further 20 minutes, then the product is filtered, washed with acetone, then with ethyl ether and finally is evaporated to dryness under vacuum at room temperature.

Yield: 7.5 g (86.7%); TLC (CH₂Cl₂:CH₃OH:CH₃COOH 70:20:10 v/v/v): Rf=0.7.

### g) Preparation of (−)-5,6-dihydroxy-2-methylaminotetralin hydrochloride

41.4 g of dry AlCl₃ (310.4 mol), 230 ml of toluene and 20.0 g of (−)-5,6-dimethoxy-2-methylaminotetralin hydrochloride (77.6 mol) under mild dry nitrogen stream and stirring, are refluxed at 80° C. for 4 hours, then cooled to room temperature to quench the reaction with ice-water (about 1000 ml total). The aqueous phase is separated and evaporated under vacuum at about 80° C. The resulting whitish solid is triturated with 750 ml of absolute ethanol, filtered and dried at 60° C.

Yield: 16.1 g (90%); TLC (CHCl₃:CH₃OH:CH₃COOH 80:15:5 v/v/v): Rf=0.15.

### h) Preparation of (−)-(S)-5,6-diisobutyroyloxy-2-methylaminotetraline hydrochloride

15 g of (−)-(S)-5,6-dihydroxy-2-methylaminotetralin are suspended in 20 ml of trifluoroacetic acid. The resulting suspension is brought to 20° C. and 20 g of isobutyroyl chloride are added under stirring. The mixture is heated at 85° C. and refluxed for 60 minutes. The solution is cooled to 50° C. and distilled under vacuum to completely remove the trifluoroacetic acid.

The resulting oily residue is taken up with 100 ml of methyl-t-butyl ether, cooled to 20° C., then the solution is saturated with gas hydrochloric acid through slow bubbling in the stirred mass, keeping the temperature at about 20° C. After about one hour, the product starts precipitating as a white crystalline solid. The solution is cooled to 15° C., washed with 50 ml of methyl-t-butyl ether and dried under vacuum at 60° C.

Yield: 22.8 g (95% in mol); m.p.: 205-208° C.; [α]_{D}: −46.8 (c=1%, H₂O); e.e.(GC-MS): >95%.

### EXAMPLE 4

### Preparation of (+)-(R)-5,6-diisobutyroyloxy-2-methylaminotetralin hydrochloride (CHF 1800) Through Enantioselective Synthesis

The procedure described in Example 3 is followed, except for the enantioselective step described in the following.

### Preparation of (−)-2-methoxycarbonylamino-4-(2,3-dimethoxyphenyl)-butyric acid

A suspension of 3-methoxycarbonylamino-5-(2,3-dimethoxyphenyl)-2,5-dihydrofuran-2-one (0.5 g, 1.7 mmol) and rhodium (S,S)EtDiPhos(COD)OTs complex (1.5 micromol) in 70 ml of previously degassed methanol is hydrogenated in a Parr apparatus (P=30 psi, T=20° C.) under stirring for two hours. The mixture is filtered, then the solution is evaporated under vacuum.

Yield: 0.5 g; Conversion: 95%; e.e.(NMR):95%.

### EXAMPLE 5

### Receptor Affinity of CHF 1024 Enantiomers

The affinity of the enantiomers for adrenergic and dopaminergic receptors was tested by binding studies in cerebral and peripheral tissues. The results are reported in Table 1 compared with CHF 1024 racemate in terms of inhibition constant (Ki) expressed as nanomolar concentration (nM).

TABLE 1

| | RECEPTOR | CHF 1024 | CHF 1870 | CHF 1869 |
|---|---|---|---|---|
| SUBSTRATE | SUBTYPE | Ki (nM) | Ki (nM) | Ki (nM) |
| Rat striatum | Central | 13000 | 6800 | 7400 |
| [³H]-SCH23390 | DA₁ receptor | | | |
| Rat striatum | Central | 290 | 120 | 2400 |
| [³H]-spiperone | DA₂-receptor | | | |
| Rat cerebral cortex | Central | 16000 | 15000 | 21000 |
| [³H]-prazosin | α₁-receptor | | | |
| Rat cerebral cortex | Central | 260 | 130 | 1600 |
| [³H]-rauwolscine | α₂-receptor | | | |
| Rat lung | β₁-receptor | 45000 | 40000 | 90000 |
| [³H]-(−)dihydro- | β₂-receptor | 1500 | 1000 | 1400 |
| alprenolol | | | | |

The results evidence that the affinity of the laevo enantiomer (CHF 1870) for DA₂-dopaminergic and α₂-adrenergic receptors is respectively about 20 and 10 times higher than that of the dextro enantiomer (CHF 1869), whereas the affinity for the other receptors is substantially the same.

Also compared with the racemate, CHF 1870 evidences higher affinity toward DA₂ and α₂ receptors with consequent lower risk of involvement of other receptor components, which are not necessary for the intended therapeutical activity.

### EXAMPLE 6

### Activity of CHF 1024 Enantiomers in Isolated Tissue Preparations

The pharmacological activity of the enantiomers toward the same receptors was also tested in isolated tissue preparations. The results are reported in Table 2 compared with CHF 1024 racemate, in terms of potency (pD₂=−log EC₅₀) and effectiveness (α). EC₅₀ is the concentration inducing 50% of the maximal response and is expressed in mols/liter (M).

TABLE 2

| | | CHF | CHF | CHF |
|---|---|---|---|---|
| TEST | | 1024 | 1870 | 1869 |
| Electrically stimulated rabbit | pD₂ | 7.6 | 8.3 | 4.2 |
| rectococcigeus muscle | | | | |
| Inihibition of contraction | α | 0.96 | 0.91 | 0.87 |
| Pre-synaptic DA₂ receptor stimulation | | | | |
| Perfused rabbit ear artery | pD₂ | 9.0 | 9.6 | <5.0 |
| Inhibition of electrically-induced | α | 0.94 | 0.92 | 0.92 |
| contraction | | | | |
| Pre-synaptic DA₂ and α₂ receptor | | | | |
| stimulating activity | | | | |
| Electrically driven guinea-pig left | PD₂ | 6.5 | 5.7 | 5.4 |
| atria | | | | |
| Increase of contractile strength | | | | |
| β₁-receptor stimulating activity | α | 0.81 | 0.91 | 0.83 |
| Guinea-pig tracheal strips contracted | pD₂ | 6.6 | 6.4 | 6.6 |
| with carbachol | | | | |
| Relaxing effect | α | 0.96 | 0.97 | 0.97 |
| β₂-receptors stimulating activity | | | | |

The results evidence that in the rabbit rectococcigeus muscle and in rabbit ear artery, which are preparations particularly rich in DA₂-dopaminergic and α₂-adrenergic receptors, the (−)-(S)-enantiomer is at least 10000 times more potent than its optical antipode. Conversely, the (+)-(R) enantiomer has β-agonist profile with poor activity on α2 and DA₂ receptors.

### EXAMPLE 7

### Activity of CHF 1024 Enantiomers on the Anaesthetized Rat

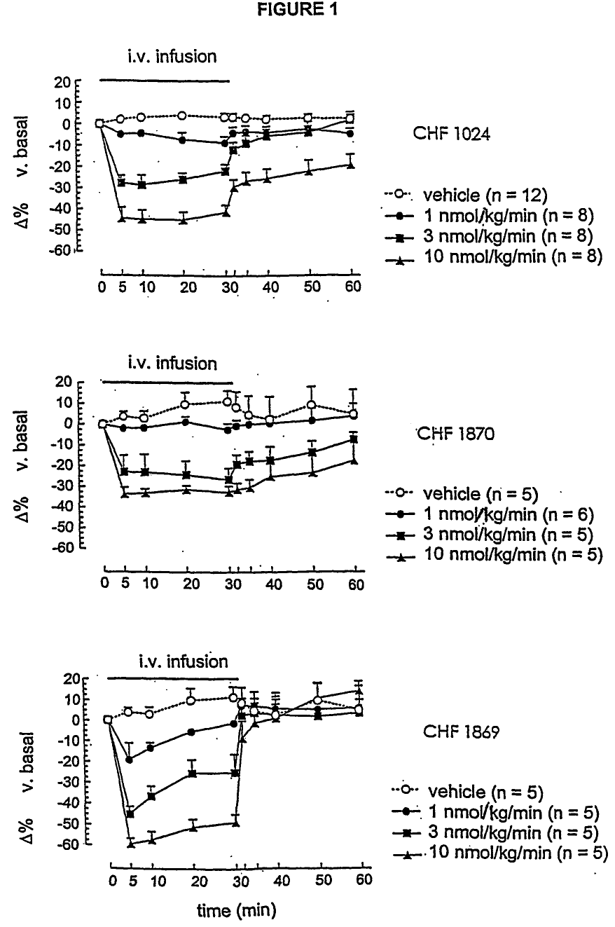

In anaesthetized normotensive rats with recording of the arterial pressure, the effects induced by intravenous infusion for 30 min of CHF 1024 enantiomers compared with the racemate were evaluated. Control animals only received the vehicle. The results are reported in FIG. 1 as mean values and standard error.

CHF 1870 induced a dose-dependent reduction of the arterial pressure which was maintained even after infusion was discontinued, which is consistent with the selectivity for pre-synaptic receptors observed in vitro.

On the contrary, the rapid onset and disappearance of the hypotensive effects induced by CHF 1869 agree with a relatively major contribution of the β₂-receptors in its action.

### EXAMPLE 8

### Activity of CHF 1024 Enantiomers in Conscious Spontaneously Hypertensive Rats

The effects induced by the enantiomers and by the racemate were also determined also in conscious spontaneously hypertensive rats, in which arterial systolic and diastolic pressure and heart rate were recorded by a telemetric system. This system consists in applying a telemetric detector in the abdominal aorta, thereby continuously recording the parameters during 24 hours while the animals are freely moving inside their cages, and avoiding any interference by the researcher. The compounds were administered by continuous infusion through subcutaneous osmotic minipumps at doses of 3 and 6 nmol/kg/min for 7 days, corresponding to about 1 and 2 mg/kg/day, respectively. In the case of the racemate, treatment was prolonged for 14 days.

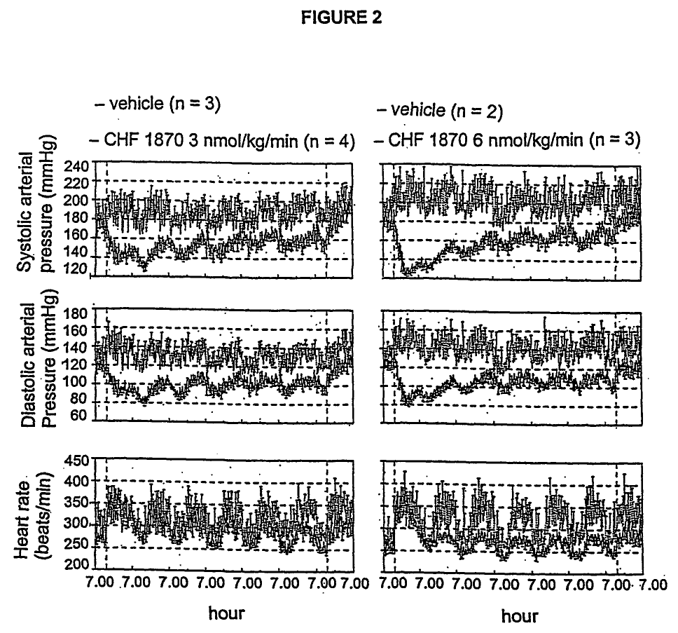

Control animals only received the vehicle. The results concerning the effects of CHF 1870, CHF 1869 and CHF 1024 are reported respectively in FIGS. 2, 3 and 4 as mean values and standard errors. The black bars indicate the treatment period.

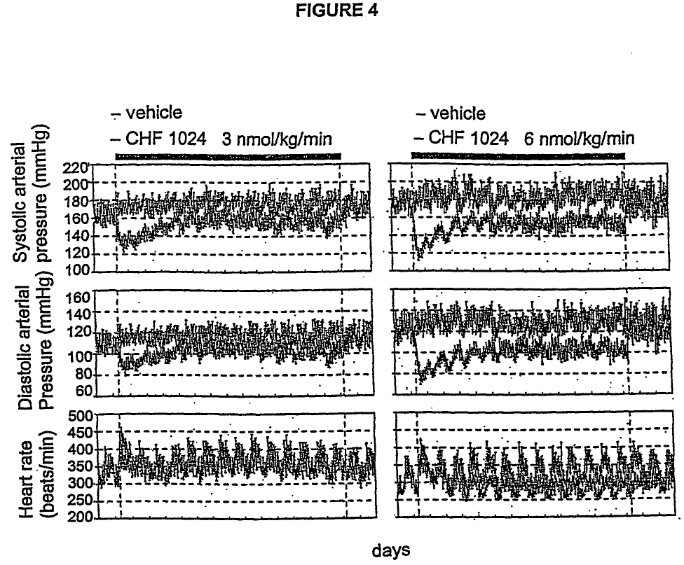

The administration of 3 nmol/kg/min of CHF 1870 (FIG. 2) induced greater reduction of heart rate compared with the racemate (FIG. 4). Furthermore, recovery to basal values for both pressure and heart rate took place more slowly. More particularly, said dose induced 20-30 mmHg reduction of arterial pressure and reduced heart rate by 30-40 beats/minute (about 10%). The administration of the higher dose caused stronger, more rapid reduction of heart rate (about 70 beats/minute).

This effect can be particularly beneficial in the treatment of patients suffering from hypertension and/or congestive heart failure.

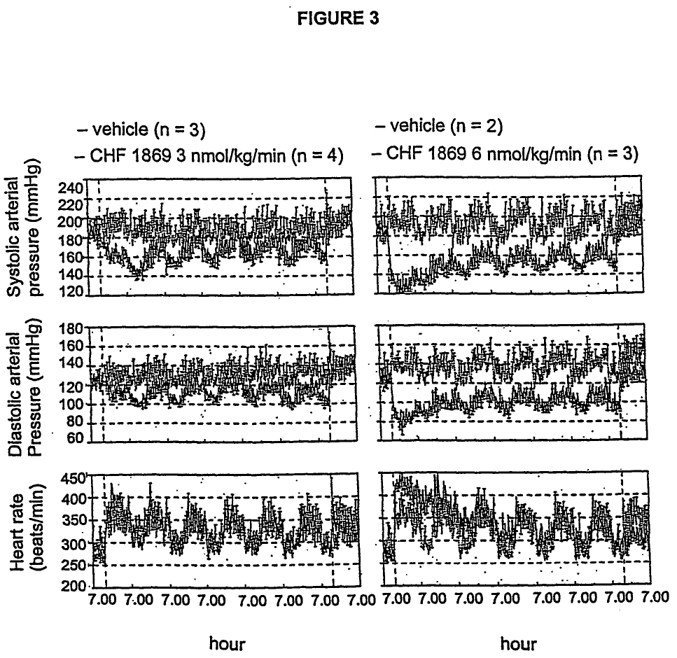

Administration of 3 nmol/kg/min of CHF 1869 induces a slight, although noticeable, hypotensive effect but no reduction of heart rate (FIG. 3). At the higher dose, the hypotensive response induced by CHF 1869 is comparable to that caused by its optical antipode; on the other hand, no reduction of heart rate is observed, which even increases during the first 2-3 days of treatment. Recovery of arterial pressure to basal values is faster than with CHF 1870, as it is observed almost immediately after interruption of the treatment.

1. Enantiomers of the compounds of formula (I) 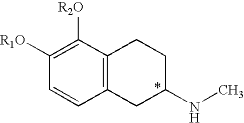
wherein R₁ and R₂, which can be the same or different, are hydrogen or a C₁-C₄ acyl group, and the pharmaceutically acceptable salts thereof, as medicaments.

2. Enantiomers as claimed in claim 1 characterized by optical purity ranging from 95% to 100%.

3. Enantiomers of 5,6-diisobutyroyloxy-2-methylaminotetralin as claimed in claim 1 or 2.

4. Compositions for the treatment of heart failure containing (−)-(S)-5,6-diisobutyroyloxy-2-methylaminotetraline or the pharmaceutically acceptable salts thereof in combination with suitable excipients.

5. Compositions as claimed in claim 4 in the form of patches for the transdermal use.

6. Compositions for the treatment of acute hypertensive crisis or of pathologies characterized by poor vascularization of the lower limbs, such as peripheral obliterans arteriopathy, containing (+)-(R)-5,6-diisobutyroyloxy-2-methylaminotetralin or the pharmaceutically acceptable salts thereof in combination with suitable excipients.

7. A process for the preparation of the optically active forms of 5,6-hydroxy-2-methylaminotetralin and esters thereof, which comprises the following steps:
a) condensation of 4-(2,3-dialkoxyphenyl)-2-ketobutenoic acid with a short chain (C₁-C₄) alkyl carbamate to give 5-(2,3-dialkoxyphenyl)-3-alkoxycarbonylamino-2,5-dihydrofuran-2-one;
b) catalytic reduction in stereoselectivity conditions of the condensation product to give one of the two enantiomers of 4-(2,3-dialkoxyphenyl)-2-alkoxycarbonylaminobutyric acid;
c) intramolecular cyclization to give one of the two enantiomers of 5,6-dialkoxy-2-alkoxycarbonyl-amino-1-tetralone;
d) reduction of the keto group to give one of the two enantiomers of 5,6-dialkoxy-2-alkoxycarbonyl-aminotetraline;
e) N-methylation of 5,6-dialkoxy-2-alkoxycarbonylaminotetralin to give one of the two enantiomers of N-methyl-5,6-dialkoxy-2-alkoxycarbonylaminotetralin;
f) hydrolysis of the alkoxycarbamic group and deprotection of the catechol group to give one of the two enantiomers of 5,6-dihydroxy-2-methylamino-tetralin;
g) optional esterification with suitable acylating agents.
